(19) United States
(12) Patent Application Publication
(10) Pub. No.: US 2021/0038458 A1
(43) Pub. Date: Feb. 11, 2021

(54) Sleep Enclosure Systems

(71) Applicant: Standish Lee, New York, NY (US)

(72) Inventor: Standish Lee, New York, NY (US)

(21) Appl. No.: 16/989,873

(22) Filed: Aug. 10, 2020

Related U.S. Application Data
(60) Provisional application No. 62/884,672, filed on Aug. 8, 2019.

(51) Int. Cl. A61G 10/00; A61G 99/00; A61L 2/10; A47C 21/04; H05K 9/00

(52) U.S. Cl. CPC A61G 10/005; A61G 99/00; A61L 2/10; A47C 21/042; A61L 2202/14; A61L 2202/121; A61L 2202/122; A61L 2202/20; A61L 2202/11; H05K 9/0003

(57) ABSTRACT

Sleep pods have automatically openable doors which open in response to the detection of hazardous conditions outside the sleep pod. Integrated disinfecting lights disinfect the interior of the sleep pod between uses.

### RELATED APPLICATION DATA

This application claims the benefit of U.S. Provisional Patent Application Ser. No. 62/884,672, filed Aug. 8, 2019, which is hereby incorporated by reference.

Sleep enclosure systems and apparatus for monitoring and enhancing sleep are disclosed. The disclosed sleep enclosures are sometimes referred to herein as sleep pods or as The Enclosure.

### BACKGROUND

Various types of sleeping pods have previously been disclosed. Such sleeping pods have not provided sufficient safety features for users. There is therefore, a need for a sleeping pod which provides enhanced safety and other desirable features for users.

### BRIEF DESCRIPTION

The various embodiments of sleep pods disclosed are suitable for people living in high density areas-such as New York city or Tokyo-who have limited living space, as well as people desiring privacy while inside the pod. The disclosed embodiments are also useful for people with sleeping difficulties who want enhanced sleep monitoring, people with Chronic Obstructive Pulmonary Disease (COPD) or emphysema who do not want to wear a mask or nasal cannula, people who want to be able to monitor their children's sleep and people concerned about the environment

As used herein, the term “The Enclosure” is used to indicate any one of the disclosed embodiments of sleep pods. As suggested by its name, The Enclosure forms an enclosure which is at least large enough to accommodate one or more persons with mattresses large enough to accommodate all occupants. The enclosure is selectively openable by the users and, according to one embodiment, opens automatically in response to one or more external alarms.

One version of The Enclosure comprises an outer shell which is preferably formed of materials containing no or low levels of volatile organic compounds (VOCs). For example, the outer shell may be formed of aluminum or steel sheet metal, a honeycomb panel of aluminum or steel sheet metal or of fiberglass.

The Enclosure is acoustically insulated, preferably with a sustainable material such as a high-density recycled rubber composite, cork and/or felt lining on the interior of the shell to isolate the interior from exterior sound.

The Enclosure is thermally insulated with a sustainable material such as cork, closed cell soy-based insulation, recycled cotton and/or Aerogel, to isolate the interior temperature from the exterior temperature.

A preferred embodiment of The Enclosure serves as a Faraday Cage, blocking electromagnetic fields (EMF) and radio frequency interference (RFI), and is electrically grounded. The aluminum sheet metal surrounding the entirety of the enclosure creates a faraday cage when connected to a ground. The ground will be an insulated cable connected at one end to the aluminum sheet metal enclosure and on the other end to a ground in an existing electrical receptable.

A compact air conditioning system for heating and cooling is installed outside of the acoustically and thermally insulated interior, from which conditioned air is delivered to the interior of The Enclosure. The air conditioning distribution is designed so that noise from the air conditioner itself is reduced. This will be accomplished by the location of the air conditioner, which is separated from the occupied interior space by a wall that has high density rubber composite, cork and/or felt lining on the interior. It is also accomplished by the configuration of the air distribution ducts, which are configured to avoid any 90-degree offsets in the flow of the air. The plenum from which the air is distributed also preferably has a dampening material applied to it such as peel and stick butyl or foam strips.

A hardwired sensor positioned in the interior of The Enclosure monitors the interior temperature for thermostats linked to the heating/air conditioning system.

A compact oxygen compressor or carbon scrubber is installed outside of the acoustically and thermally insulated interior. The oxygen compressor or carbon scrubber delivers highly oxygenated air to the interior of The Enclosure though the same opening and plenum as the conditioned air or through a separate opening proximate the end intended for a user's head when in a sleeping position and an exhaust system or vent is located proximate the end intended for the user's feet. The Enclosure is fire resistant due to the sheet metal construction.

The Enclosure has an exterior emergency detection sensor that identifies sounds commonly associated with fire or other life-threatening conditions, such as fire alarms, smoke alarms, carbon monoxide alarms, carbon dioxide alarms or the like, and triggers an audible and/or visible alarm within the interior of The Enclosure. At least one microphone is located on the exterior of the enclosure and it is set to be actively receiving sounds when the enclosure is occupied, i.e. when interior sensors detect at least one occupant. For example, a signal indicating that a user is inside of the enclosure will be provided by an IR sensor, a button that is pushed on the interior by the user, and/or the opening and closing of the door. The microphone is linked to computer hardware and software (collectively referred to as the “controller”) which recognizes common fire alarm, smoke alarm and carbon dioxide alarms and has voice recognition capability to recognize calls for, “HELP,” shouts of, “POLICE,” “PD,” and/or other emergency notifications which can be programmed into the computer system via a user interface by the user. When any of these emergency sounds are detected by the exterior microphone, the door latch will release and the door, which is on hydraulic piston(s), will automatically open. Additionally, the lights on the interior of the enclosure will be triggered to their brightest setting and will be begin to flash in a strobe-like alarm effect and the speakers will play an alarm which will sound similar to other emergency notification alarms. The controller is also selectively configurable to send an alert relating to the emergency condition to governing agencies emergency responders.

One embodiment of The Enclosure also interprets human distress signals such as yelling, crying, or screaming occurring outside the Enclosure and triggers the interior alarm.

One embodiment of the enclosure includes an external baby monitor which can be placed in a separate room and connected to the sleep pods computer through a Wi-Fi connection. This allows the sounds that are picked up from the external monitor to be played on the speakers that are within the sleep enclosure.

One embodiment of The Enclosure allows the user to silence the interior alarm.

The interior is equipped with a carbon dioxide (CO2) monitoring device, which triggers an alarm if CO2 levels in the occupied interior of the enclosure fall above those that are considered safe.

One embodiment of the enclosure includes an emergency auto-open capability where any of the before-stated alarm triggers cause the enclosure to automatically open, after which the occupant can immediately leave. A signal is sent to a latch or magnetic lock to open at which point the hydraulic pistons open the door. The Enclosure in a similar fashion as one might leave a traditional bed. A preferred embodiment of The Enclosure includes a backup battery to power the internal alarms and automatic opening feature in the event of a power outage.

The mattress in the Enclosure can comprise a 100% natural latex mattress. A wool cover is preferably implemented for fire rating as opposed to a typical mattress cover which receives its rating through chemical treatments.

The interior is wired for lighting which may be used to awaken the user. The lighting is preferably “Circadian Lighting” and the user inputs their location and the color temperature is automatically approximated for her area/region. Circadian Lighting is used as light therapy to aid in sleep.

A preferred embodiment includes a disinfecting LED light to clean the interior of the box. The user can set these disinfecting LED lights to automatically clean the interior of The Enclosure for a predetermined duration, e.g. 20 minutes, when The Enclosure is not occupied, or the user can manually activate cleaning when desired. Commercially available products by cleanslateUV (of Toronto, Ontario) and Sterilray (of Somersworth, N.H.) use ultraviolet lights in the germicidal range (between 200 nm and 320 nm) to disinfect small items, surfaces, and air. Germicidal LEDs are commercially available by manufacturers such as waveform lighting. This preferred embodiment includes germicidal LED strip lights on the interior faces of the walls of The Enclosure or at interior corners. Each strip contains approximately 20 LEDs per foot length, and it is estimated that approximately 6 feet of LED strips will be required at minimum. Germicidal LEDs by waveform lighting are capable of delivering a dose of 180 J/m2 in 15 minutes at a distance of 12 in and require a 12V DC power supply.

Sleep monitoring is implemented in one embodiment in a similar fashion to Polysomnography. The sensors for monitoring will be integrated into the enclosure itself (within the thickness of the walls, top, bottom or mattress. Electroencephalography (EEG) monitoring in Polysomnography is typically performed with electrodes applied to the human head. The Enclosure will utilize contactless EEG sensors that are installed in the mattress underneath the heads resting position or in the walls of the enclosure at a location closest to the heads resting position. Electromyography EMG is typically monitored with electrodes applied onto the human body, specifically the legs because sleep studies check for restless leg syndrome. The Enclosure monitors for leg movement using the IR sensor which can detect when there is physical movement. The computer software will determine when the legs move and distinguish this movement from rolling over in your sleep. Breathing and air flow in Polysomnography is typically monitored with a sensor at the nose (nasal cannula) to measure air flow or a strain sensor on the abdomen or ribcage. The enclosure will measure breathing and air flow through the IR sensor which is mounted to the top of the enclosure and/or a differential pressure sensor within the enclosure. If two people occupy the enclosure the differential pressure sensor will not be able to distinguish between them and therefore will not be effective for individual monitoring. Snoring is monitored through a microphone on the interior of the enclosure.

Monitoring on the interior is hard wired to the central computer on the exterior of The Enclosure, outside of the Faraday cage. The computer includes Wi-Fi and Bluetooth capabilities. Controls for all features noted herein are hard wired to the exterior of The Enclosure, because they will be blocked by the Faraday cage, and accessed by the user's phone or an exterior monitor. An application to synthesize the sleep monitoring information will be made available to the user. Controls for opening and closing, setting and adjusting alarm, adjusting temperature, setting and adjusting sound, and setting and adjusting aromas will be hardwired to the interior. None of these controls will have blue light and if they are backlit, they will have an automatic shutoff within 10 seconds of use.

A preferred embodiment of The Enclosure also includes a massage feature for physical therapy to aid in sleep, a vibrating feature for wake and/or physical therapy to aid in sleep, a screen for reading without back lighting, the ability to open into a flat bed, an enclosure for two people, digital assistant communication though the microphone and computer, voice activated control adjustments though the microphone and computer, stack-ability, an option for an earthing sheet/grounding mat and cryo-therapy.

One embodiment of The Enclosure comprises more affordable options without a custom mattress, certain sensors or physiological monitoring and auto opening devices.

### DETAILED DESCRIPTION

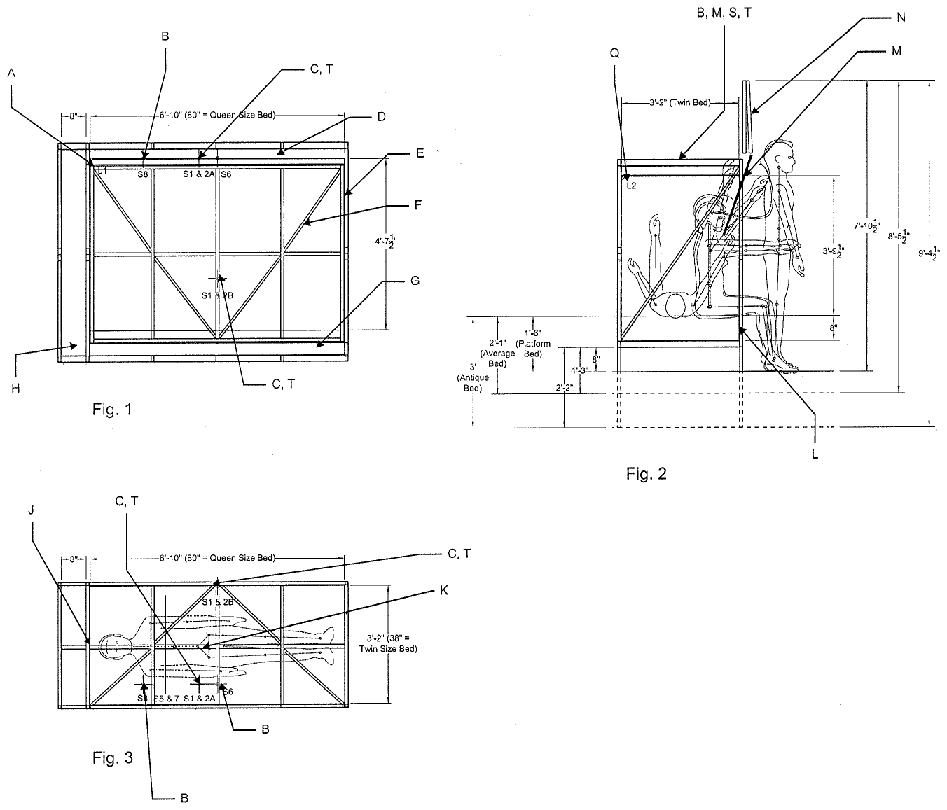

FIGS. 1, 2 and 3 illustrate and embodiment of The Enclosure designed for single occupancy.

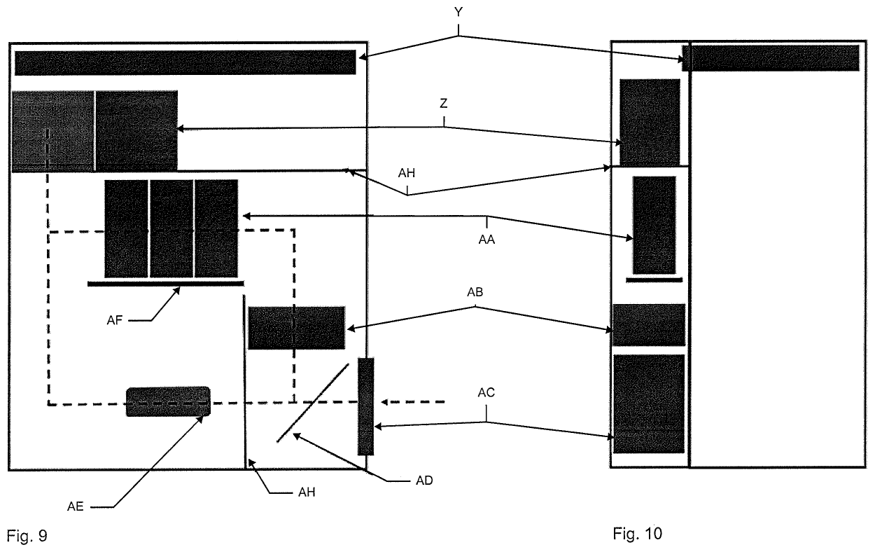

FIG. 1 is a section drawing illustrating the structure (E) of the closed door (N) of The Enclosure as well as the structure (E) of the adjacent walls, floor, and roof. The diagonal elements of the door are cross bracing (F). The illustrated wall includes exemplary dimensions of the occupiable space of The Enclosure (6 ft 10 in by 4 ft 7½ in). Also indicated in FIG. 1 is a microphone (B), CO2 monitors (C), temperature sensors or thermometers (T), as well as a plenum (D) for air delivery and return. The Sin-wide space shown is intended to contain components such as the backup battery and computer (H) among others. See FIGS. 9 and 10 for more details.

FIG. 2 is a section drawing illustrating a vertical wall of The Enclosure showing an open door (N), microphone (B), carbon monoxide detector (M), smoke detector (S), temperature sensor or thermometer (T), disinfecting LED (Q), and a latch or magnetic lock (L). FIG. 2 uses anthropometric figures of representative sizes and proportions as references.

FIG. 3 is a top-down plan view of The Enclosure and shows microphones (B), CO2 monitors (C), EEG monitors for brain activity (J), IR sensor (K), and temperature sensors or thermometers (T).

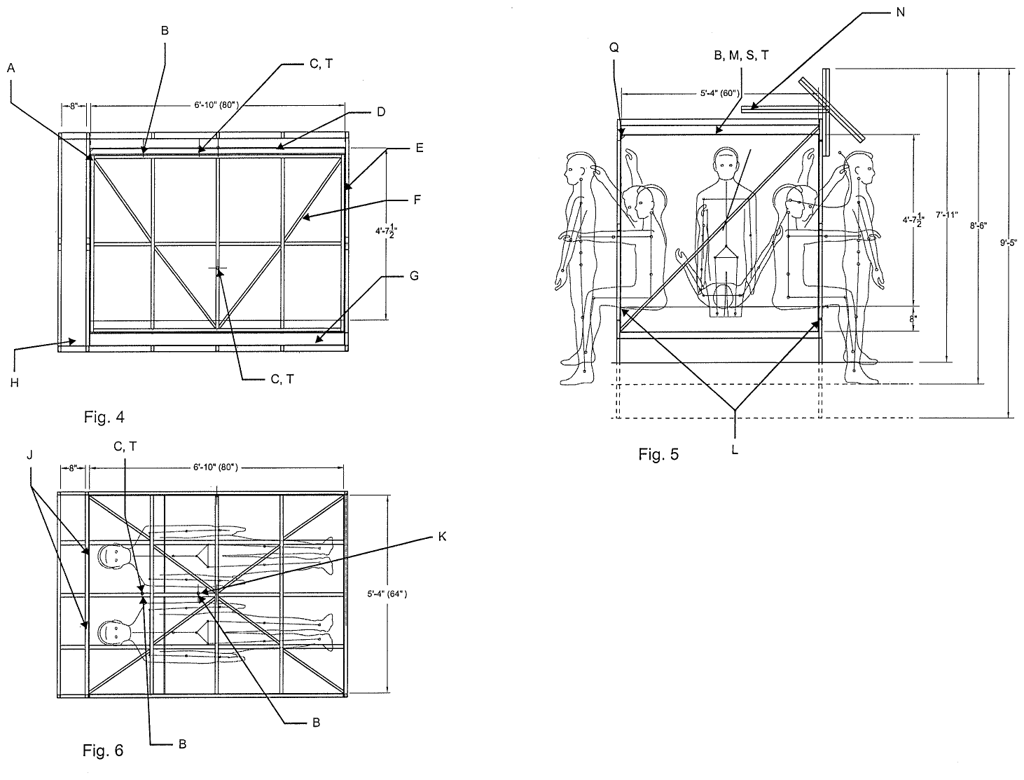

FIGS. 4, 5 and 6 illustrate an embodiment of The Enclosure designed for double occupancy.

FIG. 4 is a section drawing illustrating the structure (E) of the closed door (N) of The Enclosure as well as the structure (E) of the adjacent walls, floor, and roof. The diagonal elements of the door are cross bracing (F). The illustrated wall includes exemplary dimensions of the occupiable space of The Enclosure (6 ft 10 in by 4 ft 7½ in). Also indicated in FIG. 4 is a microphone (B), CO2 monitors (C), temperature sensors or thermometers (T), as well as a plenum (D) for air delivery and return. The Sin-wide space shown is intended to contain components such as the backup battery and computer (H) among others. See FIGS. 9 and 10 for more details.

FIG. 5 is a section drawing illustrating a vertical wall of The Enclosure showing an open door (N), microphone (B), carbon monoxide detector (M), smoke detector (S), temperature sensor or thermometer (T), disinfecting LED (Q), and a latch or magnetic lock (L). FIG. 2 uses anthropometric figures of representative sizes and proportions as references.

FIG. 6 is a top-down plan view of The Enclosure and shows microphones (B), CO2 monitors (C), EEG monitors for brain activity (J), IR sensor (K), and temperature sensors or thermometers (T).

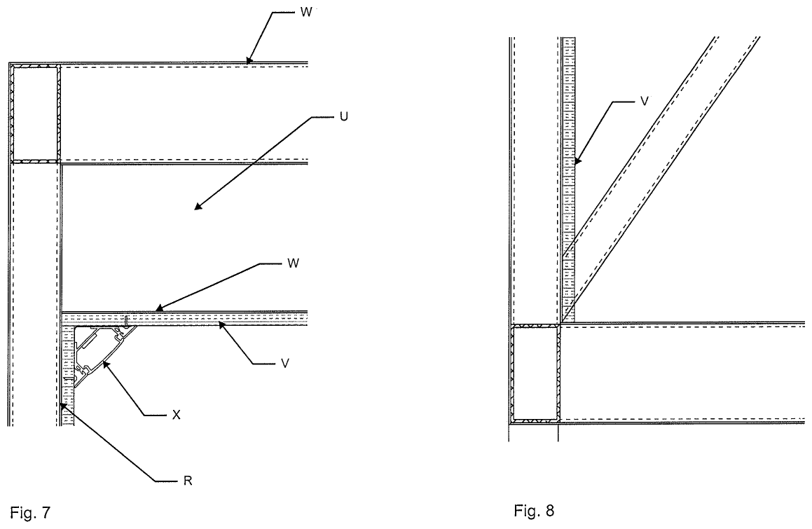

FIG. 7 shows a vertical section detail through The Enclosure with a typical corner connection detail. FIG. 7 shows high density rubber composite (R), insulation (U), felt lining (V), sheet metal (W), and LED lighting (X).

FIG. 8 shows a plan section detail with a typical corner connection detail. FIG. 8 indicates the felt lining (V).

FIG. 9 shows a diagram of components located within the Sin space indicated in FIGS. 1, 3, 4 and 6. FIG. 9 indicates a drip pan (AF), a CO2 scrubber or oxygen compressor (AE), and baffles (AH) FIG. 10 shows a diagram similar to FIG. 9, but from a view rotated 90 degrees from the view shown in FIG. 9.

FIGS. 9 and 10 show a plenum for air delivery and return (Y), a fan and sound attenuator (Z), heating a cooling coils (AA), a HEPA filter (AB), and an intake louver (AC).

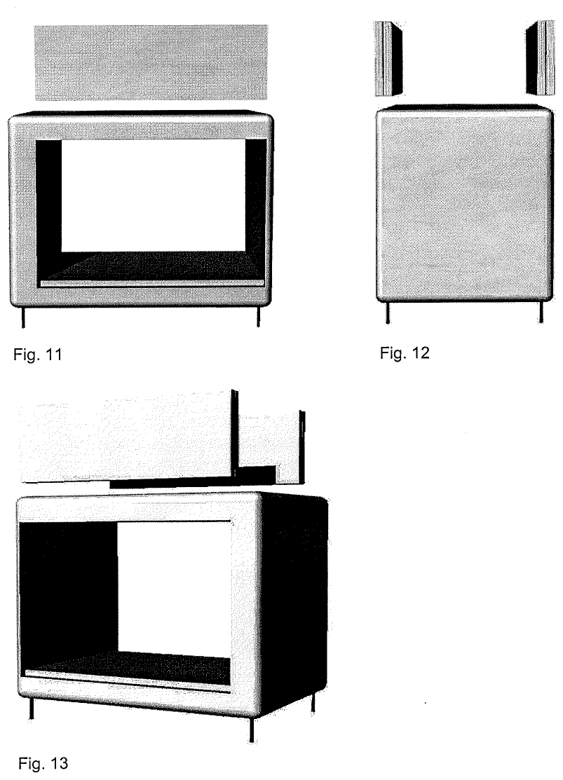

FIGS. 11, 12 and 13 show various axonometric views of The Enclosure.

For purposes of illustration, one example of a version of the Enclosure is described below. This illustrated example has the following wall composition from interior to exterior; ¼″ felt lining on the interior of the enclosure, ½″ aerogel, ⅛″ recycled rubber composite, 20 gauge aluminum sheet, 2″ of closed cell soy insulation between aluminum framing members, and an outer layer of 20 gauge aluminum sheet metal which is enameled or powder coated on the exterior surface. As noted above, one version of The Enclosure comprises an outer shell which is preferably formed of materials containing no or low levels of volatile organic compounds (VOCs). For example, the outer shell may be formed of aluminum or steel sheet metal, a honeycomb panel of aluminum or steel sheet metal to reduce the weight of the assembly or of fiberglass.

The present invention (The Enclosure) allows for energy use reduction during nighttime hours, which comprise one third of human lives, because heating and cooling is required for a smaller volume of space, i.e. the interior of The Enclosure rather than an entire room, apartment or home. The disclosed embodiments allow users to sleep in rooms with significant exterior noise. Space requirements are also be reduced because people are more readily able to sleep in the same room with those who snore at nighttime. Therefore, a need exists within the sleep industry and the greater world for a sleep enclosure such as this invention (The Enclosure).

1. A sleep pod comprising:
a base,
at least one selectively movable door which movable between at least a closed position and an open position, said door forms an enclosed interior with said base when said door is in said closed position;
at least one mattress disposed in said interior;
an actuatable, automatic door opener for automatically moving said door to said open position in response to an indication of a hazardous condition outside said enclosed interior.

2. A sleep pod according to claim 1 further comprising at least one sensor for detecting said hazardous condition outside said enclosed interior.

3. A sleep pod according to claim 2 wherein said sensor detects at least one of high carbon dioxide levels, smoke, carbon monoxide and heat.

4. A sleep pod according to claim 1 further comprising at least one microphone for detecting said sounds outside said enclosed interior and transmits sound signals to a computer which analyzes said sound signals for similarity to predetermined sound signals which are indicative of a hazardous condition, said computer sends an opening signal to said automatic door opener if a sound signal indicative of a hazardous condition is received by said computer.

5. A sleep pod according to claim 4 wherein said predetermined sound signals comprise signals corresponding to the sounds of at least one of smoke alarms, carbon monoxide alarms, carbon dioxide alarms, burglar alarms or a human voice shouting.

6. A sleep pod according to claim 1 further comprising an audible and/or visible interior alarm which is broadcast in response to an indication of a hazardous condition outside said enclosed interior.

7. A sleep pod according to claim 1 wherein said base and said door form a Faraday cage around said enclosed interior.

8. A sleep pod comprising:
a base,
at least one selectively movable door which movable between at least a closed position and an open position, said door forms an enclosed interior with said base when said door is in said closed position;
at least one mattress disposed in said interior; and
a disinfecting LED light source which disinfects at least a portion of said enclosed interior.

9. A sleep pod according to claim 8 wherein said disinfecting light source comprises at least one LED.

10. A sleep pod according to claim 8 further comprising a sensor for detecting when occupants have egressed said enclosed interior and activating said disinfecting LED light source for a predetermined time after occupants have egressed said enclosed interior.
